(19) United States
(12) Patent Application Publication
(10) Pub. No.: US 2009/0149749 A1
(43) Pub. Date: Jun. 11, 2009

(54) Method and system for synchronized playback of ultrasound images

(75) Inventor: Nicolas Heron, New York, NY (US)

Correspondence Address: Patent Administrator;Proskauer Rose LLP, One International Place, Boston, MA, 02110-2600, US

(73) Assignee: Imacor, Uniondale, NY (US)

(21) Appl. No.: 12/291,761

(22) Filed: Nov. 12, 2008

Related U.S. Application Data
(60) Provisional application No. 60/987,083, filed on Nov. 11, 2007.

(51) Int. Cl. A61B 8/00; A61B 5/0402

(52) U.S. Cl. 600/437; 600/509

(57) ABSTRACT

A method and system for synchronizing playback of ultrasound images based on user-defined parameters for concurrent and synchronous display. The method comprises defining corresponding intervals of first and second cardiac cycles captured by ultrasound imaging, associating such ultrasound images for substantially synchronized playback, and applying a selected playback speed or time interval for concurrently displaying such images.

### CROSS REFERENCE TO RELATED APPLICATION

This application claims the benefit of U.S. provisional application No. 60/987,083, filed Nov. 11, 2007, the disclosure of which is incorporated by reference herein.

### TECHNICAL FIELD

The present invention relates to a method and display system for concurrently displaying ultrasound images, and more particularly for concurrently displaying two or more echocardiogram cineloops that are synchronized over selectable intervals between editable markers.

### BACKGROUND

Ultrasound imaging of the heart, or echocardiography, has proven to be a particularly useful tool for cardiovascular studies and diagnoses of cardiovascular disease. Echocardiography uses standard ultrasound techniques to image two-dimensional slices of the heart. All of the events of a cardiac cycle can be recorded separately by cineloop, e.g., sequential images displayed at a relatively high frame-rate to provide a multidimensional depiction of the movement of a heart over time.

Electrocardiography is another useful diagnostic tool. An electrocardiogram (ECG or EKG) is a graphical representation of heart activity produced by a noninvasive transthoracic electrocardiograph, which records the electrical activity associated with the cardiac cycle of the heart over time. The cardiac cycle generally refers to all or any of the events related to the flow of blood that occur from the beginning of one heartbeat to the beginning of the next heartbeat. Every beat of the heart involves three major stages: atrial systole, ventricular systole and complete cardiac diastole. The cardiac cycle is co-ordinated by a series of bioelectrical impulses that are produced by specialized heart cells found within the sino-atrial node and the atrioventricular node.

A typical ECG waveform of a heartbeat (or cardiac cycle) includes a P-wave, a QRS complex and a T-wave. Of these various components, the QRS complex or R-wave has a dominant amplitude feature. The R-wave generally is the portion of the ECG waveform having the steepest slopes and the sharpest peaks. In a normal cardiac cycle, the peak of an R-wave usually indicates the end of diastole and the start of systole. Generally, R-wave detection algorithms are used to process ECG waveforms and detect R-wave peaks. To label detected R-waves, markers can be placed at the location of R-wave peaks. Such markers can be placed, for example, automatically by a graph-searching algorithm in the waveform or manually by a user of the imaging and display system.

In practice, an ultrasound cineloop can be saved in a digital format to be later played back on a display system. During playback, an ultrasound cineloop can be synchronized, over one or more cardiac cycles, with a corresponding ECG waveform recorded during the approximately same duration as the cineloop, so that timing of the cineloop is aligned with the timing of the ECG waveform. A time-synchronized display typically displays an image captured at about the same time as a point indicated on a corresponding ECG waveform.

For a variety of medical applications, it is useful to simultaneously display two or more cineloops, each of which representing a different cardiac cycle (or corresponding portions thereof) to facilitate side-by-side comparisons of the ultrasound images. A comparison between the cineloops of the human heart at time t₁ and time t₂ can provide valuable information for diagnosing and/or treating cardiovascular disease. For example, the comparative information from episodic assessment can be used to evaluate the efficacy or effectiveness of a course of treatment and/or to monitor the heart's reaction to administration of treatment. In echocardiography, recorded cineloops are often reviewed offline by playing back at the acquisition speed, for example, at a playback speed or rate of 50 frames-per-second. However, a meaningful comparison between multiple cineloops typically requires synchronization based on the cardiac cycle. Current methods for synchronizing cineloop playback tend to rely on computational designation of cardiac cycles by R-wave peaks, which can be unreliable for a number of reasons. For example, R-waves in a patient's ECG waveform might be atypical, and may be missed or falsely recognized by applicable algorithms, resulting in erroneously labeled cardiac cycles. Furthermore, current imaging and display systems rely on synchronizing a portion of the first full cardiac cycle from one cineloop with a corresponding portion of the first full cardiac cycle of another, and displaying the images based on the acquisition speed of one of the cineloops, and provide little flexibility to a user to select intervals of interest or reference intervals, or to control the playback speed. Therefore, a need remains for improved usability and flexibility for ultrasound imaging and display systems, and more particularly for improved methods for synchronizing playback of ultrasound cineloops based on a cardiac cycle and observations of the cardiac cycle, e.g. as determined by echocardiogram data.

### SUMMARY

The concepts described herein provide methods and systems to improve accuracy and flexibility of synchronized playback of cineloops, in particular via user interface. In one aspect, the invention provides a method for concurrently and synchronously displaying user-selected intervals of a first and a second ultrasound image loop over a selected, adjustable or variable playback speed or playback time interval. The method comprises accessing a first loop having a series of sequential ultrasound images correlated with a first reference signal over a first time interval at a first acquisition speed, the first loop capturing at least a first cardiac cycle of a heart; and accessing a second loop comprising a series of sequential ultrasound images correlated with a second reference signal over a second time interval at a second acquisition speed, the second loop capturing at least a second cardiac cycle of the heart. The method further comprises selecting a first interval on the first loop having a start point and an end point, the first interval comprising at least a subset of the sequential ultrasound images and representing at least a portion of the first cardiac cycle; and selecting a second interval on the second loop, the second interval comprising at least a subset of sequential ultrasound images and representing a portion of the second cardiac cycle corresponding to the portion of the first cardiac cycle of the first interval. According to this embodiment, the method further comprises associating the first interval and the second interval by synchronized concurrent display over a selectable playback time interval. In one aspect, the methods of the invention comprise concurrently displaying images associated with the first interval and images associated with the second interval.

In various embodiments, one or both of the first and second reference signals are electrocardiogram waveforms correlated with electrical activity of the heart. In some embodiments, the first and/or second reference signal are independent of any electrical activity of the heart.

In some embodiments, the first and/or second loop comprise an indexed image stored on an image storage medium and may be selected from one or more indexed images stored on such image storage medium. In another embodiment, the first and second loop can be, for example, separate segments of a single loop. In an exemplary embodiment, the first or second loop comprises a plurality of consecutive cardiac cycles, and the first or second cardiac cycle, as the case may be, is selected from the plurality of consecutive cardiac cycles. In an another embodiment, the first and second loop each comprises a plurality of consecutive cardiac cycles, and both the first and second cardiac cycles are selected from the respective plurality of consecutive cardiac cycles.

In some embodiments, the first interval can be an interval of interest and the second interval can be a reference interval corresponding to the interval of interest. First and second consecutive ECG markers determine the start point and end point, respectively, for an interval. An interval may be selected via a user interface, for example, by positioning a playhead or curser between two consecutive markers. In some embodiments, the start and/or end point can represent a preselected event in the first cardiac cycle. For example, the start point can represent an R-wave peak and the end point can represent a consecutive R-wave peak. In some embodiments, the second interval on the second loop is selected computationally based upon the selected first interval. For example, a user may select a first interval between an R-wave designated by a first marker as a start point and another event in the cardiac cycle designated by a second marker as the end point. According to the invention, a second interval may be computationally or automatically selected to correspond to the cycle events captured by the first interval.

In various embodiments, the method further includes selecting a different first interval or selecting a different second interval and repeating the steps of associating and concurrently displaying the first interval and the second interval using the different first interval or the different second interval, as the case may be.

The method may further include selecting a playback time interval. In some embodiments, the playback time interval is selected to allow a playback speed at which the first interval or the second interval are displayed. The playback speed can be adjusted in some embodiments, for example, in real-time mode during the concurrent display of the first interval and the second interval.

In further embodiments, the method involves adding a marker at a user-designated position or modifying a computationally-designated marker, by, for example, deleting or relocating a marker.

Some aspects of the invention relate to a processing method. The method includes receiving first variable image data and first variable reference data associated with a first cardiac cycle. According to the method, a first interval is defined by a first start time and a first end time of the first cardiac cycle, based at least in part on the first variable image data or the first variable reference data. A user can use this method to select, from a set of data structures each associated with a cardiac cycle, a data structure associated with a second cardiac cycle, the selected data structure defining a second interval with a second start time and a second end time. The method further associates the first interval with the second interval by associating the respective first and second start times and the respective first and second end times. A next step of the method involves displaying the first image data and the first reference data of the first cardiac cycle and second variable image data and second variable reference data of the second cardiac cycle over a playback time interval.

In various embodiments, the first and/or second interval are adjustable, adjustable and/or variable by a user. In some embodiments, the displayed image and reference data adjust automatically in response to an adjustment of the first or second interval. The display speed of the image and reference data may be selectable, adjustable and/or variable during or before playback. In some embodiments, the playback speed is adjustable by user-defined adjustment to the first or second interval. In some embodiments, the user selects a playback speed for (i) displaying the first image and reference data, (ii) displaying the second image and reference data, and/or (iii) the playback time interval.

Another aspect relates to an interactive imaging system for concurrent display of multiple ultrasound images. The system includes a computing apparatus having a user interface, processor, memory, and an image display system. The system receives a first set of indexed ultrasound data associated over a first interval with reference data, and a second set of indexed ultrasound data associated over a second interval with reference data, each set capturing, respectively, at least a first and a second cardiac cycle of a heart (or corresponding portions of a at least a first and second cardiac cycle). The system also has a marking module in communication with the first and second sets of indexed ultrasound data for electronically designating selected images associated with the ultrasound data. The system also includes an editing module in communication with the electronic image marker module. In another embodiment, the user interface in the computing apparatus comprises a user control for implementing user-defined marker modifications or additions. According to another embodiment, the system includes an association module that associates a user-selected first interval from the first set of ultrasound data and a corresponding second interval from the second set of ultrasound data by substantially synchronizing the first and second cardiac cycles for concurrent display over a playback time interval. The association module can automatically adjust the association upon a user-implemented modification of the first interval, or the corresponding second interval. According to one embodiment, a system of the invention further comprises a display system for concurrent display of images from the first interval of the first set of ultrasound data and images from the corresponding second interval of the second set of ultrasound data.

In some embodiments, the system further comprises a playback speed selection module in communication with a user interface for determining a speed for playback display. In one example, the playback speed selection module has a real-time mode for adjusting the playback speed during playback. In another embodiment, playback speed selection module includes a scrolling speed adjustment feature for adjusting the playback speed in a real-time mode. According to this embodiment, the playback speed is dependent upon the speed of user-actuated scrolling motion.

In some embodiments, the system further includes an echocardiography probe for acquisition of ultrasound data, and an image capturing module in communication with the probe and the processor. In some instances, a electrocardiography lead is used for acquisition of reference data, and an electrocardiogram capturing module communicates with the leads and the processor.

In various embodiments, the display system has a first display window for display of images from the first interval, and a second display window for display of images from the second interval. In some embodiments, the display system has one viewing screen, and the first and second display windows are positioned for concurrent display, in a split-view mode on a single screen, such as a monitor or a liquid crystal display (LCD). In other embodiments, the display system has at least a first and second viewing screen, and the first display window is positioned on the first screen and second display window is positioned on the second screen for displaying such images in a dual-screen mode.

In some embodiments, the user interface includes a scrolling feature in association with the user control for advancing displayed images forwards or backwards, and implementing modifications and additions of the markers. The scrolling feature can be, for example, a scrollable indicator associated with and scrollable over an electrocardiogram waveform readout of the reference data.

These and other advantages of the invention will be apparent from and will be elucidated with reference to the embodiments described hereinafter.

### BRIEF DESCRIPTION OF THE DRAWINGS

FIG. 1 depicts a schematic block diagram of a system that embodies the invention.

FIG. 2 shows a representative ECG waveform.

FIG. 3 shows a representative screen shot of synchronized display of an ultrasound cineloop image (top) and an ECG waveform (bottom).

FIG. 4 shows a representative screen shot of side-by-side synchronized playback of two cineloops.

FIG. 5 shows a screen shot of a display of an ultrasound cineloop image synchronized with an ECG waveform having missed R-wave peaks.

FIG. 6 shows a screen shot of a display of an ultrasound cineloop image synchronized with an ECG waveform having mismarked R-wave peaks.

FIG. 7 shows a screen shot of a display of an ultrasound cineloop image and a false ECG waveform.

FIG. 8 shows a screen shot of a display of an ultrasound cineloop image synchronized with an ECG waveform showing the left ventricular end-diastolic area (LVEDA) reached before R-wave peak.

FIG. 9 is a flow chart illustrating an exemplary method for storing and/or displaying image data and electrical data over a playback time interval.

FIG. 10 is a flow chart illustrating an exemplary method for synchronizing two sets of image data and electrical data over a selected playback time interval.

FIGS. 11A-11B are exemplary diagrams schematically illustrating conventional methods for synchronizing cineloops.

FIGS. 12A-12B are exemplary diagrams schematically illustrating features of editable, synchronized playback methods.

### DETAILED DESCRIPTION

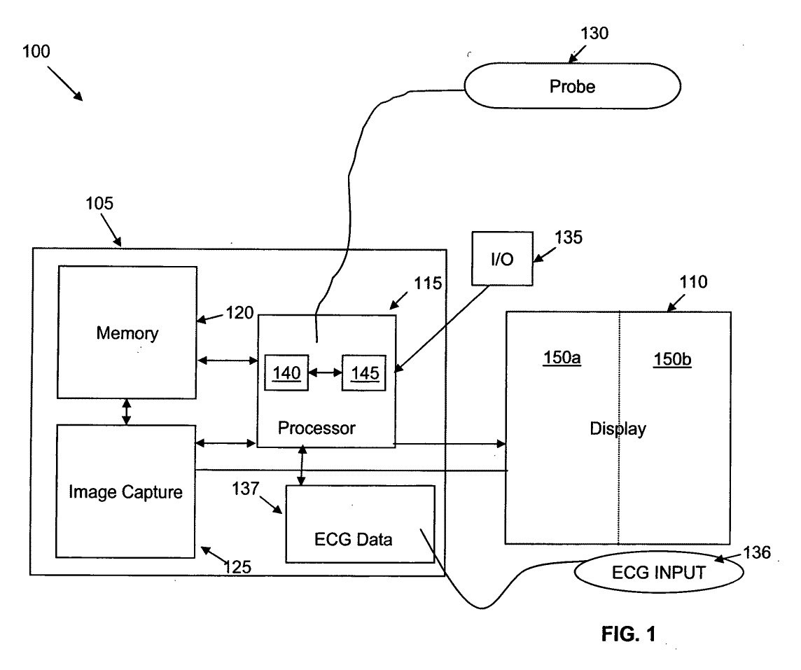

FIG. 1 depicts a schematic block diagram of a system 100 that embodies an improved system and related methods for synchronized playback of cineloops. The system 100 includes a computing apparatus or computer 105 that is in communication with a display 110. The computer 105 includes a processor 115, a memory 120, and an image capturing module 125. The image capturing module 125 is in communication with the memory 120 and the display 110 for storing and displaying images (and ECG waveforms and other data associated therewith), respectively. The memory 120 is also in communication with the processor 115. In this embodiment, more than one display is depicted. In another embodiment, single split-view (or split-screen) display capable of simultaneously displaying multiple cineloops is used. Display screens useful in the invention include, without limitation, monitors and liquid crystal displays.

The system 100 includes an imaging probe 130 in communication with the processor 115. The imaging probe 130 can be an ultrasound probe. In some embodiments, the imaging probe is a transesophageal echocardiography probe. An example of a suitable transesophageal echocardiography probe is disclosed in U.S. patent application Ser. Nos. 10/997,059 and 10/996,816, each titled “Transesophageal Ultrasound Using a Narrow Probe” by Roth et al., filed Nov. 24, 2004, the entire disclosures of both of which are incorporated by reference herein.

According to the embodiment of FIG. 1, the system also includes an ECG data module 137 for collecting and processing ECG data in a digitized format. ECG data is received at the processor 115 from an ECG input 136 via the ECG data module 137. In one embodiment, the ECG input 136 comprises standard ECG leads for use in capturing raw ECG data in a digital format. In another embodiment, the ECG input 136 is in communication with an ECG module 137 that captures and pre-processes raw ECG data, for example a standard ECG machine.

The system 100 includes an input device 135 in communication with the processor 115 for communicating information to the processor 115. For example, the input device 135 can include a mouse or pointer for use in the editing function to modify, for example, add, delete or move ECG markers associated with specific features of the cardiac cycle, as displayed on the display 110. The edited information can be used for further processing, as discussed in more detail below. The input device 135 can also control the processor 115, memory 120, image capturing module 125, ECG data module 136, and/or the display 110 by facilitating a user issuing commands the processor 115 carries out.

The image capturing module 125 collects and processes image data from the imaging probe 130 via the processor 115. For example, the image capturing module 125 can capture image data such as sequential images stored as one or more cineloops. The processor 115 can store information captured by the image capturing module 125 in the memory 120, e.g., as a data file, an image file, a cineloop file, or other type of suitable data structure. A cineloop file is a file containing multiple images, stored digitally as a sequence of individual frames. In some embodiments, the number of images or frames in a cineloop is pre-determined (e.g., 150 frames), but this is not required.

The processor 115 can include sub-modules or sub-routines for carrying out, implementing, or performing various operations and tasks. As illustrated, the processor 115 includes a calculation module 140 and an marker module 145 in communication with the calculation module 140. The processor 115 utilizes the calculation module 140 to determine, e.g., cineloop recording and/or playback speeds required to synchronize and display data. The processor 115 also includes the marker module 145 to detect and/or mark (e.g., identify) predetermined features represented in the ECG data, such as ECG peaks representing R-waves of the cardiac cycle, and to add, delete and move such markers, as discussed in more detail below.

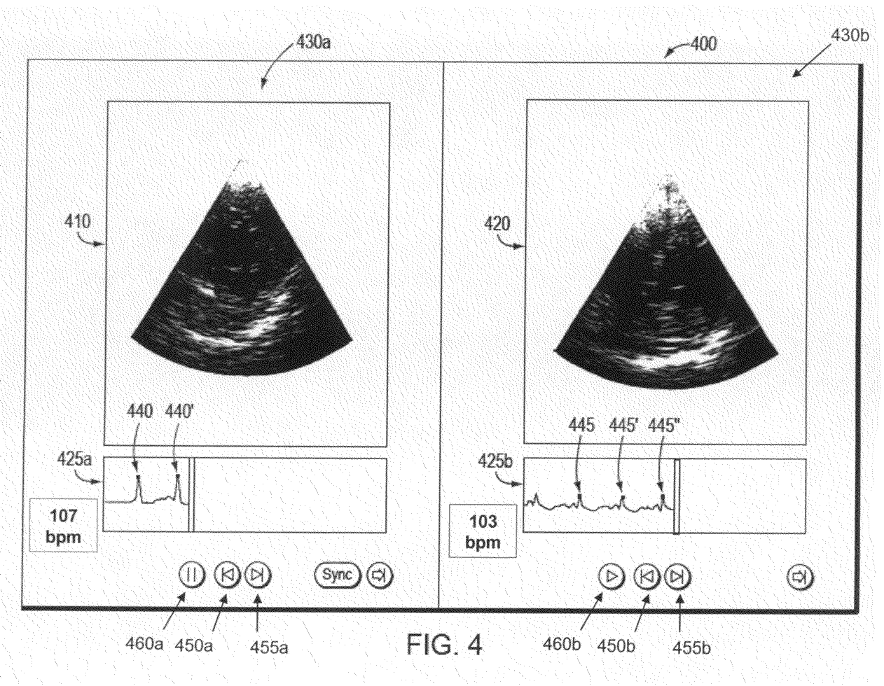

The synchronized cineloops can be viewed side-by-side on a split-screen display or on different displays. Referring to FIG. 1, display 110 may include a first area 150a and a second area 150b. The first area 150a and the second area 150b can be displayed side-by-side, with each area 150a, 150b displaying a different cineloop, e.g., as shown in FIG. 4. In some embodiments, a display shows multiple cineloops at the same or different display rates. Some embodiments feature display rates that are substantially the same as the real-time rate at which the data was acquired (e.g., data acquisition rate). According to one aspect, the display rate is represented as a percentage, or other multiplier or factor applied to the acquisition rate of the cineloop. In another aspect, the display rate is represented by a rate at which sequential frames of the cineloop are displayed, and referred to in terms of frames-per-second. In one embodiment, one or more of the cineloops are displayed at a rate that is different than the acquisition rate. For example, one or more of the cineloops may be displayed at a rate that has been calculated in order to synchronize a cardiac cycle displayed in the cineloop with a cardiac cycle displayed in a different cineloop.

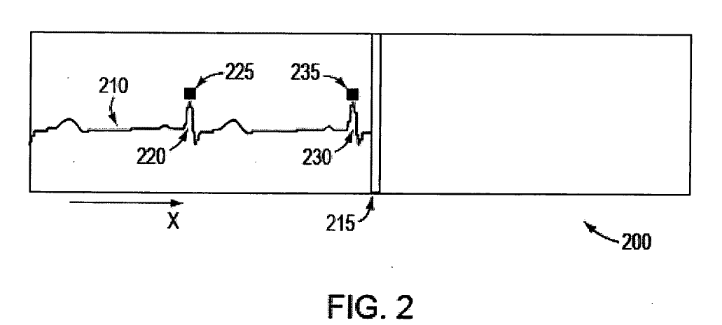

The invention contemplates, in one aspect, using an R-wave detection algorithm (e.g., operating on the processor 115) to process the ECG data and place at least a first and a second marker at consecutive R-wave peaks in order to set off or define one or more intervals. In this embodiment, the first ECG marker designates the start point of a cardiac cycle, and the second ECG marker designates the end point of the cardiac cycle. FIG. 2 shows a representative display 200 of an ECG waveform 210 having a first R-wave peak 220 and a second R-wave peak 230. The display 200 includes an ECG index 215 that moves along the waveform 210 in the direction of the X-axis. The index ECG 215 traces the time evolution of the waveform 210. In the waveform 210, R-wave peaks are marked by black square marker 225, 235 on each respective peak 220, 230. The markers 225, 235 can be associated with the peaks 220, 230 via user input or a peak-finding algorithm (e.g., R-wave detection algorithm). In practice, a user may select the interval between markers 225 and 235 as a first or second interval according to the invention.

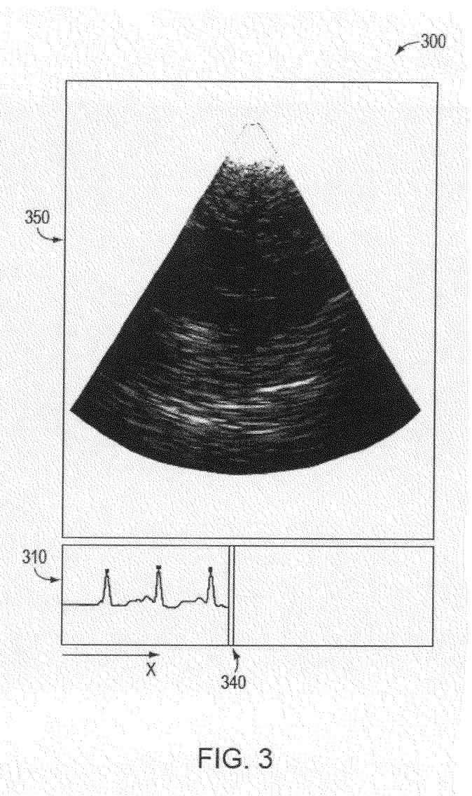

According to one aspect of the invention, an ECG waveform is used as reference for designating the placement of the marker. To determine the ultrasound image that correlates in time to the placement of the ECG marker, an ECG waveform that is time-synchronized with an ultrasound cineloop is used. FIG. 3 shows a representative screen shot of time-synchronized display 300 of an ECG waveform 310 and an ultrasound cineloop image 350. An ECG index 340 indicates a time correspondence between the cineloop image 350 and the ECG waveform 310, as the ECG index 340 moves along the X-axis as time elapses. The ECG index 340 can be used to indicate the point in time on the ECG waveform 310 that corresponds to the displayed image 350 from a cineloop. Methods for verifying the time-synchronization of ECG data with ultrasound data are disclosed, for example, in U.S. Publication No. 2008/0177181, the entire disclosure of which is incorporated by reference herein.

According to one embodiment, a system is provided that includes a module for time-synchronizing reference data generated by an ECG machine and image data generated by ultrasound machine. After the timing relationship between the two machines has been established and verified, the timing correlation can be calculated and/or transformed for subsequent times by tracking the amount of time elapsed in both systems, and by associating and/or synchronizing the elapsed time or the subsequent time (e.g., end time or a later time). Only one frame of an ultrasound image is typically displayed at any given time. An ECG waveform displays data over a period of time (e.g., a number of seconds) on the same display. A suitable user interface for indicating the timing relationship between the ultrasound images and the ECG data is to assign or associate a color with a marker on the ECG waveform that corresponds to the frame of ultrasound cineloop that is being displayed at any given time. When the ultrasound image is replayed, the colorized marker would then move along the ECG waveform, e.g., along the X-axis.

In some embodiments, when the current time corresponds to a fixed position on the ECG display screen, the fixed position can be identified on a display with a vertical line (ECG index, e.g., 340 in FIG. 3). The ultrasound frame that corresponds to the data value of the ECG waveform (e.g., 310 in FIG. 3) is displayed at that fixed position at that time. Alternative user interfaces for displaying both ultrasound data and ECG data and for indicating the temporal relationship between the ultrasound data and ECG data are also possible.

The synchronized playback function enables a user to review a first interval and at least one corresponding second interval selected from different cineloops in a synchronized fashion. Each cineloop can include at least two consecutive ECG markers, one representative of the start of a interval and one representative of an end of the interval. The two different cineloop can be, for example, separate segments of a single cineloop. In some embodiments, a software algorithm synchronizes the cineloop playback using the first two consecutive ECG markers the algorithm encounters as it progresses through each cineloop. If, for example, the cineloop frames do not advance after being loaded, these frames can correspond to the first two ECG markers associated with the cineloop.

The synchronized playback is depicted in FIG. 4, which shows a representative screen shot of side-by-side synchronized playback of cineloop images 410, 420. The cineloops 410, 420 are synchronized to the respective ECG waveforms 425a, 425b, e.g., based on corresponding cardiac cycles represented by the cineloops 410, 420 and ECG waveforms 425a, 425b. Each cineloop is associated with an ECG waveform having R-wave peaks marked by square ECG markers 440, 440′, 445, 445′, 445″. The cineloop 410 represents a real-time beats-per-minute (bpm) of 107, and the cineloop 420 represents a real-time bpm at 103. In some embodiments, the playback rate of one cineloop is modified to synchronize the cardiac cycle with a second cineloop, e.g., according to the acquisition rate. The playback rates of two or more cineloops can be all modified to synchronize playback. It is also possible for the playback rate of a cineloop to be modified to synchronize with a standard or control playback rate.

In some embodiments, the user can manually choose the playback rate. In the exemplary FIG. 4, during playback mode 460a, the forward button 450a and back button 455a function as speed controls. Clicking on forward button 450a increases the playback rate and clicking on back button 455a slows the playback. For example, the user can select to playback at a percentage of the real-time or acquisition speed, e.g., 25%, 50%, 75%, 100%, 150% or 200%. 75% means a playback rate at 75% of the original, acquisition frame rate and 200% is twice the original frame rate. In another embodiment, the user can select a playback rate at a specific frames-per-second speed without reference to the acquisition speed. When in pause mode 460b, the forward button 450b and back button 455b enable the user, for example, to step forward and backward one frame at a time. In some embodiments, the user scrolls freely through the images by moving the trackball at desired speed, using, for example, a mouse or other input device.

When a cineloop (e.g., cineloop 420) contains more than two ECG markers, the user can specify which marker will be used to begin the synchronization. In one example, the user can perform the following steps illustrated with reference to FIG. 4:
1. Left click (e.g., using a mouse or other user input device) on the portion 430b of the split view screen displaying the desired cineloop (e.g., cineloop 420);
2. When an imaging menu appears (not shown) appears, select the “Jump to Next ECG Marker” option (not shown). The “Jump to Next ECG Marker” option will, for example, advance from a first ECG marker 445 to a second ECG marker 445′, and if desired, to a third marker 445″. The user could also “drag” the cursor or ECG index to the location on the ECG wave form (e.g. 425b) where the synchronization should begin. Continue to marker where synchronization will begin, and select it as a start ECG marker (e.g.; ECG marker 445″);
3. Optionally, position an ECG marker on the second ECG waveform 425a associated with the second cineloop 410 (e.g., repeating the process of steps 1 and 2, and selecting 440′ as the start marker for ECG waveform 425a); and
4. Optionally, the user designates the end of point or time synchronization by selecting an end ECG marker.

The two start ECG markers e.g., 440′ for ECG waveform 425a, and 445″ for ECG waveform 425b, together with their respective end markers (e.g., the immediate next marker, by default), are thus used to synchronize the playback of two or more cineloops, where the first ECG marker defines the start and the second ECG marker defines the end point of a segment that is to be synchronized. In this way, allowing a user to select ECG waveform intervals allows flexibility in playback and speed control. In some embodiments, the user can place the ECG index at desired intervals, for example between two ECG markers, and the algorithm automatically synchronizes designated intervals for playback.

Many commonly-used R-wave detection algorithms can be unreliable and occasionally misinterpret an ECG waveform and/or incorrectly mark R-wave peaks and/or fail to mark true R-wave peaks. Circumstances causing this include a missing or erratic ECG signal, a signal that is too low for R-wave detection over background electrical noise, and/or misinterpretation of data that can occur with atypical ECG waveforms. Moreover, to reduce systematic, random, and/or human error, manual editing of R-wave peaks in ECG waveforms is desired for data interpretation, but this feature has not been achieved while at the same time providing sufficient user flexibility.

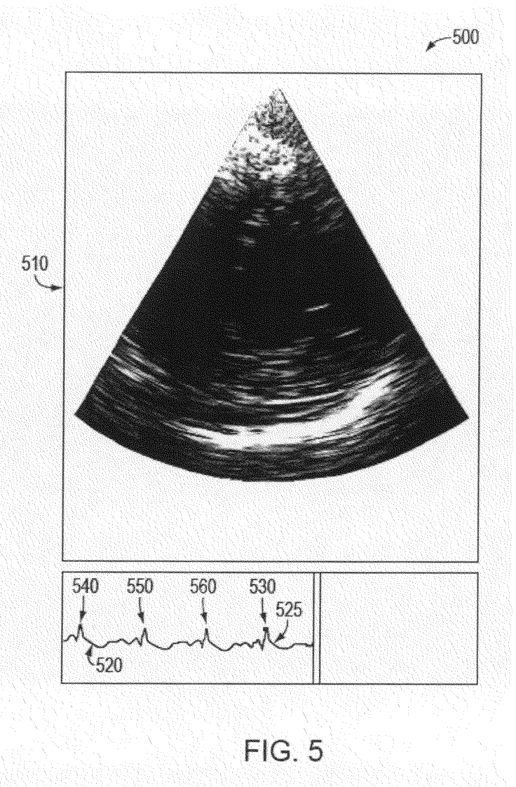

FIGS. 5-8 illustrate typical problems in ECG waveform detection and/or editing.

FIG. 5 shows a screen shot 500 of a display of an ultrasound cineloop image 510 synchronized with an ECG waveform 520 having missed R-wave peaks. A computational detection algorithm has marked one R-wave peak 525 with a square ECG marker 530 but failed to detect three others (540, 550, 560) due to background noise. Missed peaks (540, 550, 560) lead to errors or problems in synchronizing the ECG to cineloop and can lead to the diagnostic errors, e.g., one interval actually represents multiple cardiac cycles as opposed to one.

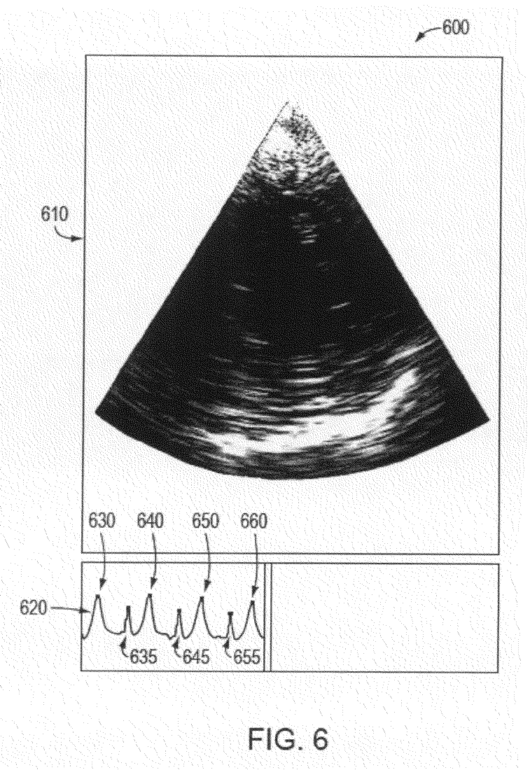

FIG. 6 shows a screen shot 600 of a display of an ultrasound cineloop image 610 synchronized with an ECG waveform 620 having missed R-wave peaks. A computational detection algorithm has marked seven R-wave peaks by markers 630, 635, 640, 645, 650, 655, 660. Some peaks have been mismarked by markers 635, 645, 655 due to false detection by the algorithm. The user can recognize and correct incorrect detection by placing the ECG index 670 at proper position on the ECG waveform 620 or repositioning the markers at the correct peaks. In FIG. 6, the interval between two consecutive markers (e.g., 645 and 650) is less than a full cardiac cycle. The user could use, for example, the system depicted in FIG. 1, to view the ultrasound images corresponding to the markers 635, 645 or 655, and correlate them to the correct part of the cineloop.

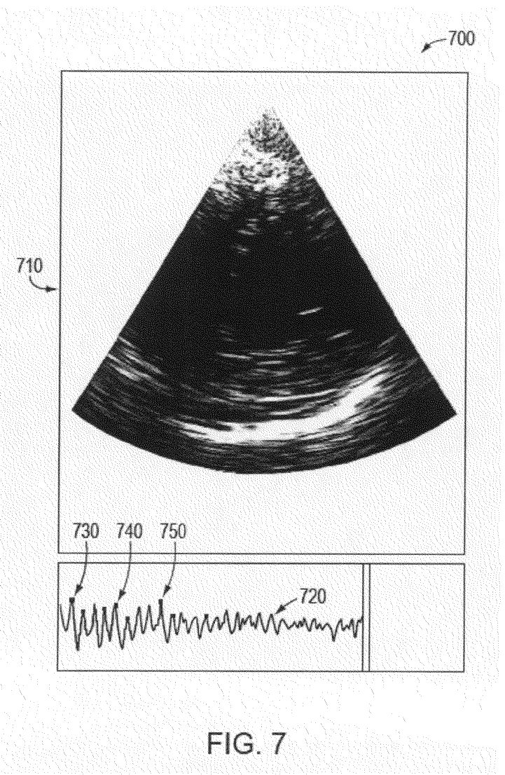

FIG. 7 shows a screen shot 700 of a display of an ultrasound cineloop image 710 synchronized with a false ECG waveform 720. That is not representative of actual electric activity of the heart. No real ECG signal was measured because of, for example, human error during recording of ECG data. An R-wave detection algorithm has marked false R-wave peaks at for example 730, 740 and 750. Synchronization of the waveform 720 with cineloop 710 would not produce meaningful data for analysis or diagnostic purposes.

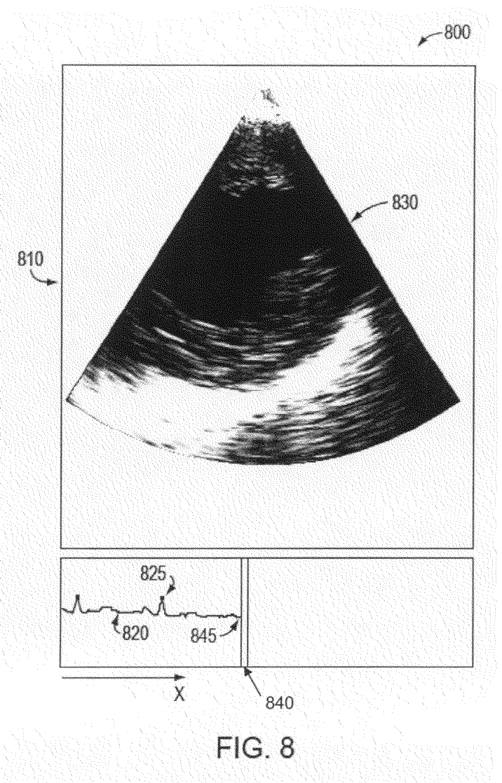

FIG. 8 shows a screen shot 800 of a display of an ultrasound cineloop image 810 synchronized with an ECG waveform 820, illustrating a left ventricular end-diastolic area (LVEDA) maximum occurrence before an R-wave peak in the ECG waveform 820. The left ventricle pumps blood into the systemic circulation. The LVEDA generally represents the total amount of blood in the left ventricle at the end of diastole. End-diastole is generally defined as the ultrasound image frame corresponding to the largest left ventricle cross-sectional area, and usually occurs immediately after the R-wave peak 825 on the ECG waveform 820. However, under certain disease conditions, the image frame corresponding to LVEDA may occur before R-wave peak in the ECG waveform 820. As for example illustrated in FIG. 8, the LVEDA image frame 830 corresponds to the ECG index at position 845 on the ECG waveform 820. As shown, the ECG waveform 820 has not yet reached the next R-wave peak at this time point. Therefore, when an R-wave detection algorithm identifies R-wave peaks in this situation, the resulted marker would not accurately indicate the end diastole and could result in a mischaracterized cardiac cycle. Using, for example, the system depicted in FIG. 1, it is possible to edit the placement of ECG markers so that each ECG marker accurately corresponds to the end diastole. Such ECG marker editing feature is described in detail hereinafter.

Additional sources of ECG marker misplacement exist. For example, R-wave detection may be less accurate in the presence of noise, such as (1) motion artifacts from body muscle depolarization and repolarization; (2) changes in contact features between the electrodes and the skin; and/or (3) changes in overall amplitude and average level of the ECG signal due to breathing or other phenomena that affect body conductance.

In embodiments where the user (e.g., ultrasound technician) determines the R-wave was not identified correctly by the R-wave detection algorithm, an ECG marker editing feature allows the user to manually set and remove markers on the ECG waveform 820. For example, as the cineloop 810 moves forward in time, the ECG index 840 progresses through the ECG waveform 820 along the X-axis, in a synchronous fashion. The ECG index 840 appears highlighted when coinciding with an ECG marker. If the ECG marker causing the highlighting is erroneous, the user can modify the marker by, for example, deleting the marker. The user can also move (e.g., via user input) to a cineloop frame (e.g. image 830) that corresponds to end diastole and manually introduce an ECG marker that was missed by the R-wave detection algorithm.

ECG markers can be entered and/or edited manually. One way for the user to edit an ECG marker is to scroll or move the index 840, while the correlated cineloop data 810 automatically adjust to the time position of the ECG waveform 820. For example, in various embodiments, the user can execute the following exemplary functions:

Adding an ECG Marker:
1. The user moves the ECG index 840 to the desired location of the ECG waveform 820 using a user input (e.g., 135 of FIG. 1);
2. The user right clicks the trackball button on the input, which triggers an interface menu;
3. The user chooses “Add ECG Marker” option from the pop-up menu. An marker is thereby associated with the waveform 820 and can be used for data processing and synchronized playback.

Deleting an ECG Marker
1. The user moves the ECG index 840 until it is highlighted over the marker the user would like to remove, using a user input (e.g., 135 of FIG. 1);
2. The user right clicks click the trackball button on the input, which triggers an interface menu;
3. The user chooses “Delete ECG Marker” option from the pop-up menu, thereby removing the marker.

In some embodiments, if all ECG markers associated with a waveform and/or a cineloop are deleted, the software will attempt to detect the R-wave peaks and associate ECG markers the next time a cineloop is loaded.

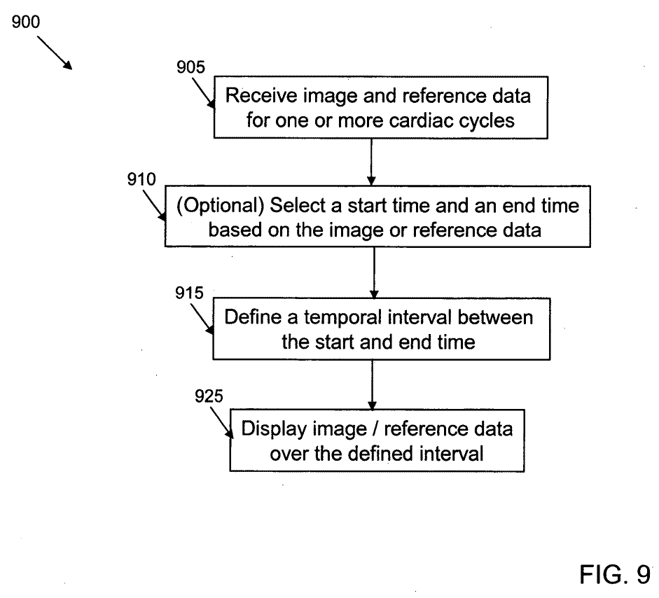

FIG. 9 is a flow chart 900 illustrating an exemplary process for storing and/or displaying image data and electrical data over a temporal interval. In general, image data and/or reference data for one or more cardiac cycles are received (step 905). Data is generally received as a data structure, e.g., from a memory or a buffer. The image and/or reference data can be recorded substantially simultaneously using, for example, the system 100 depicted in FIG. 1. In particular, the user accesses a loop comprising a series of sequential ultrasound images that are correlated with a reference signal (e.g., an ECG waveform or an electrical signal independent of the electrical activity of the heart) over a time interval at an acquisition speed. The loop generally captures or represents at least one cardiac cycle of a heart, and are accessed by selecting an indexed image from an image storage medium. The user can, for example, select separate segments of a single loop and generate independent loops.

After step 905, the user can manually select an interval based on the image or reference data (step 910). For example, the user may define an interval or an interval on the loop having a start point and an end point, where the interval comprises at least a subset of sequential ultrasound images. The interval can represent a whole or partial cardiac cycle. Step 910 allows the user to define an interval between the start and end time (step 915). The user can also select an interval (e.g., start and end time) based on the ECG data.

After step 915, the user has the option to determine whether to archive the image and/or reference data by, for example, issuing a command through an input device such as the input device 135 of the system 100 of FIG. 1, to store the image and/or reference data. If the user, in response to prompt, opts to store the data, the image and/or reference data is stored over the defined interval in for example, the memory 120 in FIG. 1. Generally, the image and/or reference data are automatically displayed (step 925) on for example, the display 110 shown in FIG. 1.

At step 910, the user can, for example, scroll forwards or backwards through the cineloop images, and associate or correlate start and/or end markers (e.g., as discussed above). The user can also move the index along the ECG waveform and determine the start and/or end markers. Under either implementation the user defines or determines a interval between the start and end time. The user can additionally indicate a preference for display options, for example in a same or a different viewing window. In some embodiments, the user can set up or select the playback speed for the synchronized image and/or reference data. The playback rate or speed can be the acquisition rate of either the first or second intervals, or some arbitrary speed. In another embodiment, a user can adjust the playback speed in a real-time mode via the user interface and a scrolling feature associated with a playback speed selection module of a system according to the invention.

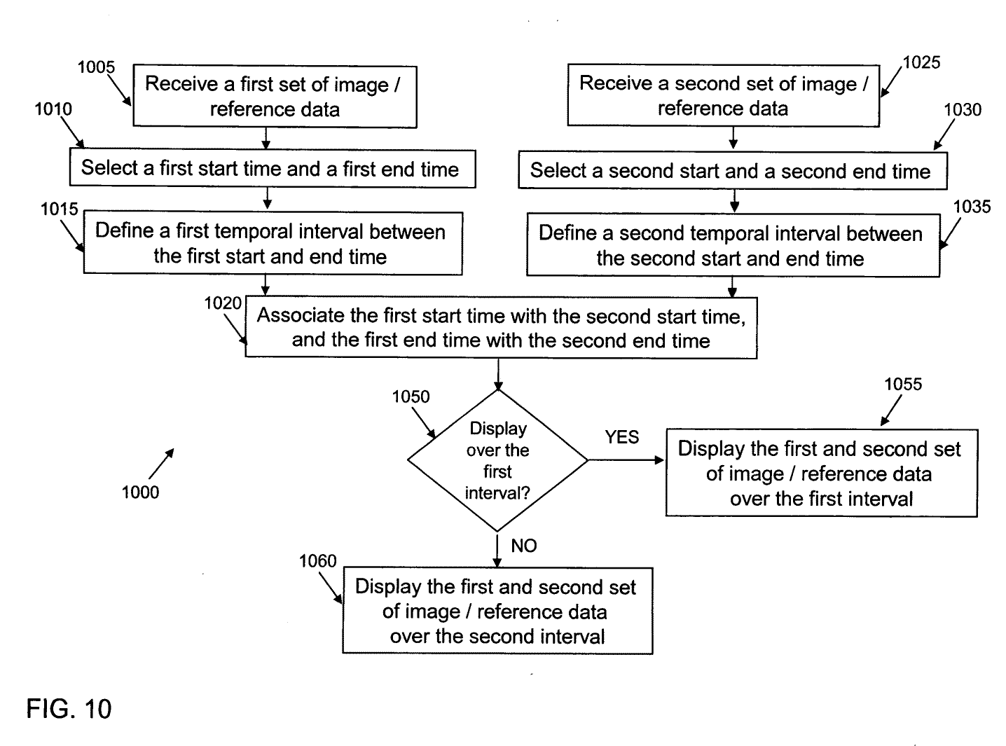

FIG. 10 is a flow chart 1000 illustrating an exemplary process for synchronizing two sets of image data and reference data over a selected playback time interval. A first and second set of variable image data and variable reference data are received by, for example the processor 115 in FIG. 1 (steps 1005, 1025). Each set of data is associated with one or more cardiac cycles. In particular, the user accesses a first and a second loop each comprising a series of sequential ultrasound images that are correlated with a reference signal (e.g., an ECG waveform or an electrical signal independent of the electrical activity of the heart) over a time interval at an acquisition speed. A user can select a first interval (step 1015) for the first data set (step 1010) and a second interval (step 1035) for the second data set (step 1030) respectively, using for example the process shown in FIG. 9. For example, the user may define a first and a second interval on the respective loop, each interval having a start point and an end point, where each interval comprises at least a subset of sequential ultrasound images. In an exemplary embodiment, the first and second loop each comprises a plurality of consecutive cardiac cycles, and the first and second cardiac cycle is selected, respectively, from the first and second plurality of cardiac cycles.

In some embodiments, the first interval can be an interval of interest and the second interval can be a reference interval corresponding to the interval of interest. In some embodiments, the starting and/or end point of an interval can designated by marker positioned, for example, at a preselected point in the first cardiac cycle. For example, the start point can represent an R-wave peak and the end point can represent a consecutive R-wave peak. In some embodiments, the second interval on the second loop is selected computationally based upon the selected first interval. In various embodiments, the user has the option to select a different first interval or select a different second interval and repeat the steps of associating and concurrently displaying the first interval and the second interval using the different first interval or the different second interval.

At step 1020 in FIG. 10, the first interval is associated with the second by associating the respective first and second start times and the respective first and second end times. In one embodiment, the first interval and the second interval are associated by aligning corresponding portions of the first and second cardiac cycles. The user may have the option to decide whether to archive the updated data by, for example, issuing a command through an input device such as the input device 135 of the system 100 of FIG. 1. The updated data may be stored in for example, the memory 120 in FIG. 1.

At step 1050, a prompt asks the user whether to display the data over the first interval. A positive command, query or request renders the two sets of image and/or reference data to be displayed over the first interval (step 1055). The first and second data sets are otherwise displayed in a synchronized fashion, over playback time interval at the user's choice (step 1060). Alternatively for step 1050 the user is prompted to select whether or not to display over the playback time interval the image and/or reference data. The user can additionally indicate any display preference. In another embodiment, the user can set up or select the playback speed for the image and/or reference data.

In various embodiments, the user may further select a different first interval and/or a different second interval and repeat the steps of associating and concurrently displaying the first interval and the second interval. It is also possible to select a different playback speed or time interval and repeat the step of concurrently displaying the first interval and the second interval. In some embodiments, the playback time interval is selected to allow a playback speed at which the first interval or the second interval are displayed. The playback speed can be adjusted in some embodiments, for example during the concurrent display of the first interval and the second interval. In further embodiments, the user can edit the computationally-designated marker, by, for example, deleting or relocating the computationally-designated marker.

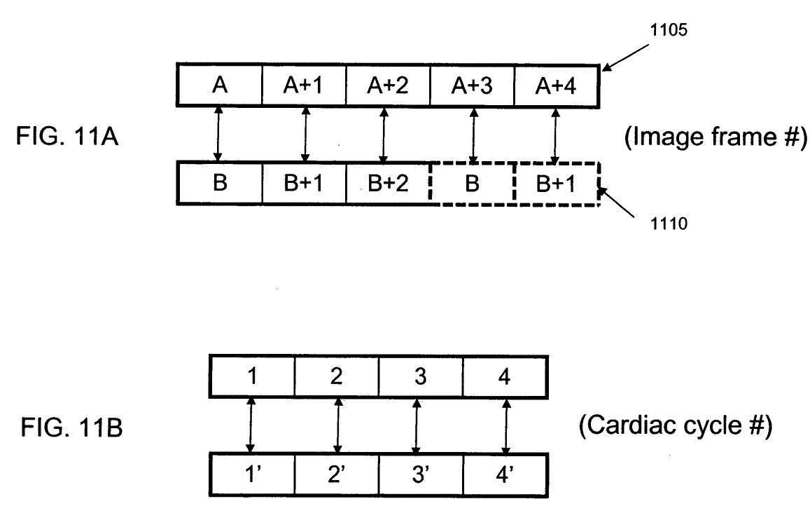

FIGS. 11A-11B are exemplary diagrams schematically illustrating conventional methods for synchronizing cineloops. FIG. 11A relates to methods for synchronizing cineloops 1105 and 1110 that capture cardiac cycles having different numbers of image frames. In this example, the speed of playback is frame-based and cannot be changed. Synchronization is achieved by repeated playing of certain frames (B and B+1) so that the number of frames viewable are the same between two cineloops 1105 and 1110. FIG. 11B shows that the order of playback as predetermined by the order of recorded cardiac cycles. No editing function is available to choose and compare cardiac cycles of interest.

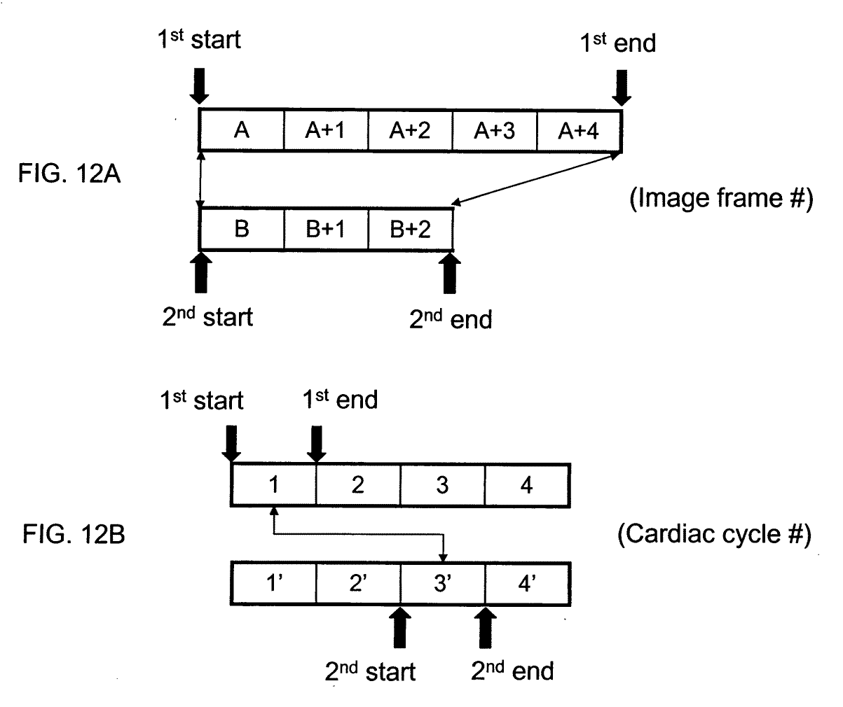

FIGS. 12A-12B are exemplary diagrams schematically illustrating features of editable, synchronized playback methods. FIG. 12A demonstrates the speed of playback is variable and can be adjusted so that the first and second intervals finish playing at substantially the same time. The user can choose either the first or the second interval as the base interval, and have both cineloops played at the speed of the base interval. For example, the method can calculate a percentage of frames played in the base interval, and impose the same percentage on the other interval. FIG. 12B shows that playback can be changed by a user adjusting the first and/or second interval. The user has the freedom to selectively place the start and/or end marker for the first and/or second interval, thereby allowing playback of selected cardiac cycles in a synchronized manner. It is therefore possible to compare, for example, selected cardiac cycles from two cineloops that are out of order.

Also contemplated herein is synchronization over multiple cardiac cycles captured in a single cineloop and corresponding ECG waveform. Methods and systems are particularly useful in this context and can be applied to the synchronization over multiple cycles of cineloops of varying lengths.

The above-described techniques can be implemented in digital electronic circuitry, or in computer hardware, firmware, software, or in combinations of them. The implementation can be as a computer program product, e.g., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps can be performed by one or more programmable processors executing a computer program to perform functions of the technology by operating on input data and generating output. Method steps can also be performed by, and apparatus can be implemented as, special purpose logic circuitry, e.g., an FPGA (field programmable gate array) or an ASIC (application-specific integrated circuit). Modules can refer to portions of the computer program and/or the processor/special circuitry that implements that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

The terms “module” and “function,” as used herein, mean, but are not limited to, a software or hardware component which performs certain tasks. A module may advantageously be configured to reside on addressable storage medium and configured to execute on one or more processors. A module may be fully or partially implemented with a general purpose integrated circuit (“IC”), FPGA, or ASIC. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules. Additionally, the components and modules may advantageously be implemented on many different platforms, including computers, computer servers, data communications infrastructure equipment such as application-enabled switches or routers, or telecommunications infrastructure equipment, such as public or private telephone switches or private branch exchanges (“PBX”). In any of these cases, implementation may be achieved either by writing applications that are native to the chosen platform, or by interfacing the platform to one or more external application engines.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communications, e.g., a communications network. Examples of communications networks, also referred to as communications channels, include a local area network (“LAN”) and a wide area network (“WAN”), e.g., the Internet, and include both wired and wireless networks. In some examples, communications networks can feature virtual networks or sub-networks such as a virtual local area network (“VLAN”). Unless clearly indicated otherwise, communications networks can also include all or a portion of the PSTN, for example, a portion owned by a specific carrier.

The computing system can include clients and servers. A client and server are generally remote from each other and typically interact through a communications network. The relationship of client and server arises by virtue of computer programs running on the respective computers and having a client-server relationship to each other.

Various embodiments are depicted as in communication or connected by one or more communication paths. A communication path is not limited to a particular medium of transferring data. Information can be transmitted over a communication path using reference, optical, acoustical, physical, thermal signals, or any combination thereof. A communication path can include multiple communication channels, for example, multiplexed channels of the same or varying capacities for data flow.

Multiple user inputs can be used to configure parameters of the depicted user interface features. Examples of such inputs include buttons, radio buttons, icons, check boxes, combo boxes, menus, text boxes, tooltips, toggle switches, buttons, scroll bars, toolbars, status bars, windows, or other suitable icons or widgets associated with user interfaces for allowing a user to communicate with and/or provide data to any of the modules or systems described herein.

### EQUIVALENTS

While the invention has been particularly shown and described with reference to specific embodiments, it should be understood by those skilled in the art that various changes in form and detail may be made therein without departing from the spirit and scope of the invention as defined by the appended claims.

What is claimed is:

1. A method for displaying an ultrasound image, the method comprising:
accessing a first loop comprising a series of sequential ultrasound images correlated with a first reference signal over a first time interval at a first acquisition speed, said first loop capturing at least a first cardiac cycle of a heart;
accessing a second loop comprising a series of sequential ultrasound images correlated with a second reference signal over a second time interval at a second acquisition speed, said second loop capturing at least a second cardiac cycle of said heart;
selecting a first interval on said first loop having a start point and an end point, said first interval comprising at least a subset of said sequential ultrasound images and representing at least a portion of said first cardiac cycle;
selecting a second interval on said second loop, said second interval comprising at least a subset of said sequential ultrasound images and representing a portion of said second cardiac cycle corresponding to said portion of said first cardiac cycle of said first interval;
associating said first interval and said second interval for substantially synchronized concurrent display of said corresponding portions of said first and second cardiac cycles over a selectable playback time interval; and
concurrently displaying images associated with said first interval and images associated with said second interval over a playback time interval.

2. The method of claim 1, wherein at least one of said first and second reference signals comprises an electrocardiogram waveform correlated with electrical activity of said heart.

3. The method of claim 1, wherein at least one of said first and second reference signals is independent of any electrical activity of said heart.

4. The method of claim 1, wherein said first loop or said second loop comprises an indexed image stored on an image storage medium.

5. The method of claim 1, wherein said first loop and said second loop comprise different indexed images stored on an image storage medium.

6. The method of claim 1, wherein said first interval comprises an interval of interest and said second interval comprises a reference interval corresponding to said interval of interest.

7. The method of claim 1, wherein said first interval is defined by a first and second consecutive markers, and wherein said first marker designates a start point of said first interval and said second marker designates and end point of said first interval.

8. The method of claim 1, wherein further comprising marking said reference data with a marker designated at least one of said start point and said end point of said first interval.

9. The method of claim 1 wherein said start point represents a preselected event in said first cardiac cycle.

10. The method of claim 1, wherein said end point represents a preselected event in said first cardiac cycle.

11. The method of claim 1, wherein said start point represents a first R-wave peak and said end point represents a second R-wave peak, and wherein said first and second R-wave peaks are consecutive.

12. The method of claim 1, wherein said first loop and said second loop are separate segments of a single loop.

13. The method of claim 1, wherein said step of selecting a second interval on said second loop is performed computationally based upon the selected first interval.

14. The method of claim 1, further comprising selecting a different first interval or selecting a different second interval and repeating said steps of associating and concurrently displaying said first and second intervals using said different first interval or said different second interval.

15. The method of claim 1, further comprising selecting a playback time interval.

16. The method of claim 1, wherein said step of selecting a playback time interval comprises selecting a playback speed at which said first interval or said second interval is displayed.

17. The method of claim 1, wherein said method of selecting a playback time interval comprising adjusting a playback speed at which said first interval or said second interval are displayed.

18. The method of claim 17, wherein said playback speed is adjusted in a real-time mode during said concurrent display of said first interval and said second interval.

19. The method of claim 7, further comprising modifying said marker.

20. The method of claim 19, wherein said step of modifying said marker comprises deleting or relocating said marker.

21. The method of claim 1, wherein said first loop comprises a plurality of consecutive cardiac cycles and wherein said first cardiac cycle is selected from said plurality of cardiac cycles from said first loop.

22. The method of claim 1, wherein said second loop comprises a plurality of consecutive cardiac cycles and wherein second cardiac cycle is selected from said plurality of cardiac cycles from said second loop.

23. A method comprising:
receiving first variable image data and first variable reference data associated with a first cardiac cycle;
selecting a first interval including a first start time and a first end time of the first cardiac cycle, based at least in part on the first variable image data or the first variable reference data;
selecting, from a set of data structures each associated with a cardiac cycle, a data structure associated with a second cardiac cycle, the selected data structure defining a second interval including a second start time and a second end time;
associating the first interval with the second interval by associating the respective first and second start times and the respective first and second end times; and
concurrently displaying the first image data and the first reference data of the first cardiac cycle and second variable image data and second variable reference data of the second cardiac cycle over a playback time interval.

24. The method of claim 23, wherein said playback time interval comprises an adjustable duration.

25. The method of claim 24, wherein said displayed image and reference data adjust automatically in response to the adjustment of the interval.

26. The method of claim 23, further comprising selecting a playback speed for said step of concurrently displaying said image and reference data.

27. The method of claim 26, wherein said step of selecting a playback speed comprises selecting (i) a playback speed for displaying said first image or reference data, (ii) a playback speed for displaying said second image and reference data, or (iii) said playback time interval.

28. The method of claim 23, wherein said image and reference data is displayed at a playback speed that is variable according to a user-defined adjustment.

29. An interactive image display system for concurrent display of multiple ultrasound images, the system comprising:
a computing apparatus comprising a user interface, processor, memory, and an image display system;
a first set of indexed ultrasound data associated over a first time interval with reference data, said ultrasound data capturing at least a first cardiac cycle of a heart;
a second set of indexed ultrasound data associated over a second time interval with reference data, said ultrasound data capturing at least a second cardiac cycle of said heart;
a marking module in communication with said first and second sets of indexed ultrasound data for electronically designating selected images associated with said ultrasound data with a marker;
an editing module in communication with said marking module and said user interface comprising a user control for implementing user-defined modifications of markers;
an association module for associating a user-defined first interval from said first set of ultrasound data and a corresponding second interval from said second set of ultrasound data by substantially synchronizing said first and second cardiac cycles, or corresponding portions thereof, for concurrent display over at a user-defined playback speed over a playback time interval, said association module automatically adjusting said association upon a user-implemented modification of said first interval or said corresponding second interval; and
a display system for concurrent display of images from said first interval of said first set of ultrasound data and images from said corresponding second interval of said second set of ultrasound data.

30. The system of claim 29, further comprising a playback speed selection module in communication with a user interface for determining said playback speed.

31. The system of claim 30, wherein said playback speed selection module comprising a real-time mode for adjusting said playback speed during playback.

32. The system of claim 29, further comprising an echocardiography probe for acquisition of ultrasound data, and an image capturing module in communication with said probe and said processor.

33. The system of claim 29, further comprising an electrocardiography lead for acquisition of reference data, and an electrocardiogram capturing module in communication with said leads and said processor.

34. The system of claim 29, wherein said display system comprises a first display window for display of images from said first interval, and a second display window for display of images from said second interval.

35. The system of claim 34, said display system comprising a viewing screen, and wherein said first and second display windows are positioned for concurrent viewing on a single screen.

36. The system of claim 34, said display system comprising at least a first and second viewing screens, wherein said first display window is positioned on said first screen and second display window is positioned on said second screen.

36. The system of claim 29, wherein said reference data comprises an electrocardiogram waveform and said user interface comprises a scrolling control feature in association with said user control for scrolling forwards or backwards through the displayed images, and implementing modifications and additions of said makers, said scrolling control feature comprising a scrollable indicator associated with and scrollable over said waveform.

37. The system of claim 31, wherein said playback speed selection module comprises a scrolling speed adjustment feature for adjusting said playback speed in said real-time mode, wherein said playback speed is correlated to a user-actuated scrolling motion.
